(19) United States
(12) Patent Application Publication
(10) Pub. No.: US 2026/0035468 A1
(43) Pub. Date: Feb. 5, 2026

(54) NEW ANTI-ITGB8 ANTIBODIES AND ITS USES THEREOF

(71) Applicants: INSTITUT NATIONAL DE LA SANTÉ ET DE LA RECHERCHE MÉDICALE, Paris (FR); CENTRE NATIONAL DE LA RECHERCHE SCIENTIFIQUE, Paris (FR); CENTRE LEON BERARD, Lyon (FR); UNIVERSITÉ CLAUDE BERNARD - LYON 1, Villeurbanne (FR)

(72) Inventors: Julien MARIE, Lyon Cedex 08 (FR); Jordane Samuel PRETO, Lyon (FR); Hélène TARAYRE, Lyon (FR)

(21) Appl. No.: 19/110,688

(22) PCT Filed: Sep. 12, 2023
(86) PCT No.: PCT/EP2023/075036

§ 371 Date: Mar. 11, 2025

(30) Foreign Application Priority Data
Sep. 12, 2022 (EP) 22306340.5

(51) Int. Cl. C07K 16/28

(52) U.S. Cl. CPC C07K 16/2839; C07K 2317/33; C07K 2317/76

(57) ABSTRACT

Among the strategies allowing cancer cells to escape the immune system, the presence of Transforming growth factor beta (TGF-β) in the tumor micro-environment (TME) is one of the most potent immunosuppressive mechanisms for many types of tumors. Thus, depleting TGF-β is regarded as a promising potent therapeutic approach to fight cancers, regardless of the type of tumor. However, the major impediment for achieving this objective is that TGF-β belongs to the few cytokines that must be activated once secreted. Moreover, a systemic targeting of TGF-β is associated with profound side effects due to TGF-β ability to sustain the homeostasis of numerous tissues. Hence, selectively targeting of TGF-β activation within the TME appears as a rational approach to boost the anti-tumor response and avoid side effects. The integrin αvβ8 expressed on Tregs specifically promotes TGF-β activation and impairs the anti-CD8 T cell response within the TME of different cancers in both mice and humans. The inventors generated anti-β8 chain of αvβ8 integrin (Itgβ8) neutralizing monoclonal antibodies which specifically bind to Tregs and selectively neutralize the ability of Tregs to activate the TGF-β. Thus, the present invention relates to isolated anti-Itgβ8 neutralizing antibodies which specifically binds to β8 chain of αvβ8 integrin (Itgβ8) expressed on Tregs, and their uses for treating cancer.

### FIELD OF THE INVENTION

The invention relates to new antibodies that specifically binds to β8 chain of αvβ8 integrin (Itgβ8), especially the β8 chain of αvβ8 integrin (Itgb8) expressed by Treg cells. These specific antibodies can be used for the therapy of cancers.

### BACKGROUND OF THE INVENTION

The tenet of tumor immunotherapy is based on the ability of the immune system to survey for malignant transformation and be efficient at eliminating cancer cells. However, solid tumors can escape from the immune system by orchestrating a micro-environment that limits an efficient anti-tumor immune response.

Transforming Growth Factor beta (TGF-β) is regarded as a key cytokine promoting immunosuppression in the tumor micro-environment (Batlle and Massague, 2019). Among the three isoforms of TGF-β (TGF-β-3), TGF-β1 is prevalent within tumors (Gao et al., 2013). This polypeptide cytokine, highly conserved in all mammals (Shull et al., 1992), impairs numerous functions of effector T lymphocytes and promotes both development and stability of CD4^{pos} Foxp3^{pos} regulatory T lymphocytes (Tregs) (Marie et al., 2006). Subsequently, the selective targeting of TGF-β signalling in T lymphocytes leads to an efficient elimination of cancer cells by effector T lymphocytes (Gorelik and Flavell, 2001) and neutralization of TGF-β-immunoregulatory effects has been thought as a promising anti-cancer therapy (Dahmani and Delisle, 2018) (Huynh et al., 2019).

However, TGF-β is one of the few cytokines secreted in an inactive form. This small latency complex is composed of the mature cytokine encircled by the latency-associated peptide (LAP), which are non-covalently associated. LAP covers all the contact sites of the mature cytokine that must interact with TGF-β receptor complexes (TGFβRI and TGFβRII) to induce TGF-β signalling, including the phosphorylation of SMAD2/3 (Travis and Sheppard, 2013). Within solid tumors, TGF-β-latent complex can be secreted by several cell types, including cancer cells, and Tregs (Batlle and Massague, 2019). Nevertheless, unlike the one produced by Tregs, TGF-b secreted by cancer cells has been shown as essential for the repression of tumor infiltrating lymphocytes (Courau et al., 2016; Donkor et al., 2011). As long as LAP maintains close contacts with the mature cytokine, the secreted latent TGF-β can be stored in the tumor micro-environment without any immune regulatory functionality. Hence, the activation of the secreted TGF-β latent complex, which involves biochemical modifications that will separate the LAP domain from the mature cytokine, is therefore indispensable for TGF-β-immune-regulatory functions in tumors. Moreover, a systemic targeting of TGF-β is associated with profound side effects due to TGF-β ability to sustain the homeostasis of numerous tissues REF. Hence, selectively targeting of TGF-β activation within the tumor micro-environment (TME) appears as a rational approach to boost the anti-tumor response and avoid side effects.

The previous work of the inventors reveals that the β8 chain of the αvβ8 integrin (Itgβ8) expressed on Tregs specifically promotes TGF-β activation selectively in the TME and impairs the anti-tumoral CD8 T cell response against different cancers in both mice and humans (Lainé et al. Nature Com 2021).

There is thus a need to develop drug targeting specifically Itgβ8 expressed by Tregs in order to restore efficient anti-tumoral CD8 T cell response blocked by TGF-β.

Previous study discloses anti-αvβ8 integrin antibodies (WO2021/151889; WO2022/057862, WO2011103490). However, these are the first generated antibodies which selectively bind to the β8 chain of αvβ8 integrin specific of Tregs, i.e the β8 chain of the αvβ8 integrin anchored in the membrane of Tregs.

### SUMMARY OF THE INVENTION

The present invention refers to an isolated anti-β8 chain of αvβ8 integrin (Itgβ8) neutralizing antibody, wherein said antibody specifically binds to β8 chain of αvβ8 integrin expressed on Tregs.

In particular, the invention refers to an isolated anti-β8 chain of αvβ8 integrin (Itgβ8) neutralizing antibody comprising:
(a) a heavy chain wherein the variable domain comprises:
   a H-CDR1 having the following sequence: G-Y-T-F-T-X₆-Y-X₈ (SEQ ID NO: 14) wherein X₆ is S or R, and X₈ is T or W;
   a H-CDR2 having the following sequence: I-N-P-S-S-G-Y-T (SEQ ID NO:2);
   a H-CDR3 having the following sequence: A-R-X₃-E-X₅-X₆-X₇-X₈-X₉-X₁₀-X₁₁-Y-X₁₃-X₁₄-X₁₅-X₁₆-X₁₇ (SEQ ID NO:15) wherein X₃ is A or none, X₅ is G or V, X₆ is L or Y, X₇ is R or Y, X₈ is A or Y, X₉ is W or G, X₁₀ is F or S, X₁₁ is A or S, X₁₃ is G or none, X₁₄ is D or none, X₁₅ is F or none, X₁₆ is D or none and X₁₇ is Y or none; and
(b) a light chain wherein the variable domain comprises:
   a L-CDR1 having the following sequence: X₁-X₂-V-X₄-X₅-X₆, wherein X₁ is Q or S, X₂ is N or S, X₄ is G or S, X₅ is T or Y, and X₆ is N or none,
   a L-CDR2 having the following sequence: X₁-X₂-S, wherein X₁ is S or R, and X₂ is A or T,
   a L-CDR3 having the following sequence: Q-Q-Y-X₄-S-Y-P-X₈-T (SEQ ID NO: 16) wherein X₄ is N or H, and X₈ is Y or L.

In particular, the invention refers to an isolated anti-β8 chain of αvβ8 integrin (Itgβ8) neutralizing antibody comprising:
(a) a heavy chain wherein the variable domain comprises: a H-CDR1 having a sequence set forth as SEQ ID NO:1 or SEQ ID NO:9; a H-CDR2 having a sequence set forth as SEQ ID NO:2; and a H-CDR3 having a sequence set forth as SEQ ID NO:3 or SEQ ID NO:10; and
(b) a light chain wherein the variable domain comprises: a L-CDR1 having a sequence set forth as SEQ ID NO:4 or SEQ ID NO: 11; a L-CDR2 having a sequence set forth as SAS or RTS; and a L-CDR3 having a sequence set forth as SEQ ID NO:5 or SEQ ID NO:12.

Another aspect of the invention refers to a method for treating cancer in a subject in need thereof, comprising administering to said subject an effective amount of the anti-β8 chain of αvβ8 integrin (Itgβ8) neutralizing antibody of the invention.

### DETAILED DESCRIPTION OF THE INVENTION

The inventors generated monoclonal antibodies against Itgβ8 chain which selectively neutralize the ability of Tregs to activate the TGF-β. Antibodies were generated by immunization of mice deficient in Itgβ8 with Tregs expressing αvβ8 integrin. The complete depletion of Itgβ8 lead to severe and fatal autoimmunity (Aluwihare, P. et al, 2009), whereas no autoimmunity was reported after selectively Itgb8 ablation in Treg cells (Worthington, J. J. et al, 2015). For the first time, the antibodies of the invention have selective tropism for Tregs and should not be associated with autoimmune side effects, in contrast to current antibodies targeting Itgβ8.

### Definition

As used herein, the term “β8 chain of the αvβ8 integrin” or “Itgβ8” has its general meaning in the art and refers to a transmembrane glycoprotein that exclusively heterodimerizes with the alpha-V chain to form the αvβ8 integrin. Integrin αvβ8 can bind to numerous extracellular matrix (ECM) proteins and is reported to be a key protein for TGF-β activation. The UniprotKB reference for Itgβ8 is β26012.

Through their αβ8 integrin, Tregs activate the inactive TGF-β produced by cancer cells, which in turn increases the levels of TGF-β signaling in effector CD8 T lymphocytes and represses their cytotoxic functions against cancer cells. Integrin αvβ8 is expressed on Tregs in vivo and exhibits a specific steric conformation that seems unique to Tregs, as explained in Lienart S. et al Science 2018.

As used herein, the term “T regulatory cells” or “Tregs” has its general meaning in the art and refers to a specialized subpopulation of T lymphocytes that act to suppress immune response, thereby maintaining homeostasis and self-tolerance. Tregs have numerous acknowledged biomarkers known in the art. In mice, Tregs express the biomarkers Foxp3 and CD4 (CD4^{pos} Foxp3^{pos} cells) in mice. In human, Tregs are often defined as CD3^{pos}, CD4^{pos}, CD25^{hi}, Foxp3^{pos}, and CD127^{lo}.

As used herein the term “antibody” or “immunoglobulin” have the same meaning, and will be used equally in the present invention. The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CHI, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs.

In the context of the invention, the amino acid residues of the antibody of the invention are numbered according to the IMGT numbering system. The IMGT unique numbering has been defined to compare the variable domains whatever the antigen receptor, the chain type, or the species (Lefranc M.-P., “Unique database numbering system for immunogenetic analysis” Immunology Today, 18, 509 (1997); Lefranc M.-P., “The IMGT unique numbering for Immunoglobulins, T cell receptors and Ig-like domains” The Immunologist, 7, 132-136 (1999).; Lefranc, M.-P., Pommie, C., Ruiz, M., Giudicelli, V., Foulquier, E., Truong, L., Thouvenin-Contet, V. and Lefranc, G., “IMGT unique numbering for immunoglobulin and T cell receptor variable domains and Ig superfamily V-like domains” Dev. Comp. Immunol., 27, 55-77 (2003).). In the IMGT unique numbering, the conserved amino acids always have the same position, for instance cysteine 23, tryptophan 41, hydrophobic amino acid 89, cysteine 104, phenylalanine or tryptophan 118. The IMGT unique numbering provides a standardized delimitation of the framework regions (FR1-IMGT: positions 1 to 26, FR2-IMGT: 39 to 55, FR3-IMGT: 66 to 104 and FR4-IMGT: 118 to 128) and of the complementarity determining regions: CDR1-IMGT: 27 to 38, CDR2-IMGT: 56 to 65 and CDR3-IMGT: 105 to 117. If the CDR3-IMGT length is less than 13 amino acids, gaps are created from the top of the loop, in the following order 111, 112, 110, 113, 109, 114, etc. If the CDR3-IMGT length is more than 13 amino acids, additional positions are created between positions 111 and 112 at the top of the CDR3-IMGT loop in the following order 112.1, 111.1, 112.2, 111.2, 112.3, 111.3, etc. (http://www.imgt.org/IMGTScientificChart/Nomenclature/IMGT-FRCDRdefinition.html)

As used herein, the term “amino-acid sequence” has its general meaning and is a sequence of amino acids that confers to a protein its primary structure. According to the invention, the amino-acid sequence may be modified with one, two or three conservative amino acid substitutions, without appreciable loss of interactive binding capacity. By “conservative amino acid substitution”, it is meant that an amino acid can be replaced with another amino acid having a similar side chain. Families of amino acids having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., glycine, cysteine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

According to the invention a first amino-acid sequence having at least 70% of identity with a second amino-acid sequence means that the first sequence has 70; 71; 72; 73; 74; 75; 76; 77; 78; 79; 80; 81; 82; 83; 84; 85; 86; 87; 88; 89; 90; 91; 92; 93; 94; 95; 96; 97; 98; 99%; or 100% of identity with the second amino acid sequence. Amino-Sacid sequence identity is typically determined using a suitable sequence alignment algorithm and default parameters, such as BLAST P (Karlin and Altschul, 1990).

According to the meaning of the present invention, the “identity” is calculated by comparing two aligned sequences in a comparison window. The sequence alignment allows determining the number of positions (nucleotides or amino acids) in common for the two sequences in the comparison window. The number of positions in common is therefore divided by the total number of positions in the comparison window and multiplied by 100 to obtain the identity percentage. The determination of the identity percentage of sequence can be made manually or thanks to well-known computer programs.

As used herein, the terms “purified” and “isolated” relate to the antibody of the invention and mean that the antibody is present in the substantial absence of other biologic macromolecules of the same type. The term “purified” as used here means preferably that at least 75% in weight, more preferably at least 85% in weight, even more preferably at least 95% in weight, and the more preferably at least 98% in weight of antibody, compared to the total weight of macromolecules present.

As used herein, the term “nucleic acid molecule” has its general meaning in the art and refers to a DNA or RNA molecule.

As used herein, the term “specifically binds to” means that an antibody only binds to the antigen of interest, as assessed using native proteins present on the surface of isolated target cells (here the β8 chain of αvβ8 integrin (Itgβ8) expressed on Tregs) and does not exhibit cross-reactivity to other antigens.

As used herein, the term “specificity” or “specifically” refers to the ability of an antibody to detectably bind an epitope presented on an antigen, such as the β8 chain of αvβ8 integrin (Itgβ8) expressed on Tregs, while having relatively little detectable reactivity with other proteins or structures (such as β8 chain of αvβ8 integrin presented on other cell types, other proteins presented on Tregs, or on other cell types). Specificity can be relatively determined by binding or competitive binding assays, using, e.g., flow cytometry assay (such as detailed in FIG. 2) s or cell-based ELISA assay.

The term “affinity”, as used herein, means the strength of the binding of an antibody to an epitope. The affinity of an antibody is given by the dissociation constant Kd, defined as [Ab]×[Ag]/[Ab-Ag], where [Ab-Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of Flow cytometry or cell-based Elisa assay.

The terms “monoclonal antibody”, “monoclonal Ab”, “monoclonal antibody composition”, “mAb”, or the like, as used herein refer to a preparation of antibody molecules of single molecular composition. A monoclonal antibody composition displays a single binding specificity and affinity for a particular epitope.

As used herein, the terms “treatment” or “treating” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, a “therapeutically effective amount” is intended for a minimal amount of active agent which is necessary to impart therapeutic benefit to a patient. For example, a “therapeutically effective amount of the active agent” to a patient is an amount of the active agent that induces, ameliorates or causes an improvement in the pathological symptoms, disease progression, or physical conditions associated with the disease affecting the patient. It will be understood that the total daily usage of the compounds and compositions of the present invention will be decided by the attending physician within the scope of sound medical judgment. The specific therapeutically effective dose level for any particular patient will depend upon a variety of factors including the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 100 mg/kg of body weight per day,

As used herein the terms “administering” or “administration” refer to the act of injecting or otherwise physically delivering a substance as it exists outside the body (e.g., the nanobody or polypeptide according to the invention) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

As used herein, the terms “combined treatment”, “combined therapy” or “therapy combination” refer to a treatment that uses more than one medication. The combined therapy may be dual therapy or bi-therapy.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different

As used herein, the term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

### 1) Antibodies of the Invention

The sequences of interest in the present application are indicated in the following Table 1:

TABLE 1
Sequences of the antibody of the invention.

| | SEQ ID NO: | Sequence |
|---|---|---|
| VH-CDR1 | SEQ ID NO: 1 | GYTFTSYT |
| (1G10G4-1G10H5) | | |
| VH-CDR2 | SEQ ID NO: 2 | INPSSGYT |
| (1G10G4-1G10H5) | | |
| VH-CDR3 | SEQ ID NO: 3 | ARAEGLRAWFAY |
| (1G10G4-1G10H5) | | |
| VL-CDR1 | SEQ ID NO: 4 | QNVGTN |
| (1G10G4-1G10H5) | | |
| VL-CDR2 | | SAS |
| (1G10G4-1G10H5) | | |
| VL-CDR3 | SEQ ID NO: 5 | QQYNSYPYT |
| (1G10G4-1G10H5) | | |
| Mab 1G10G4-1G10H5 | SEQ ID NO: 6 | QVQLQQSGAELARPGASVKMSCKASGYTFTSYTM |
| domain VH: | | HWVKQRPGQGLEWIGYINPSSGYTKYNQKFKDKA |
| FR1-CDR1-FR2-CDR2- | | TLTADKSSSTAYMQLSSLTSEDSAVYYCARAEGL |
| FR3-CDR3-FR4 | | RAWFAYWGQGTLVTVSA |
| Mab 1G10G4-1G10H5 | SEQ ID NO: 7 | DIVMTQSQKFMSTSVGDRVSVTCKASQNVGTNVA |
| domain VL: | | WYQQKPGQSPKALIYSASYRYSGVPDHFTGSGSG |
| VL FR1-CDR1-FR2- | | TDFTLTIINVQSEDLAEYFCQQYNSYPYT |
| CDR2-FR3-CDR3-FR4 | | FGGGTKLEIK |
| VH-CDR1 (9B2) | SEQ ID NO: 8 | GYTFTRYW |
| VH-CDR2 (9B2) | SEQ ID NO: 2 | INPSSGYT |
| VH-CDR3 (9B2) | SEQ ID NO: 9 | AREVYYYGSSYGDFDY |
| VL-CDR1 (9B2) | SEQ ID NO: 10 | SSVSY |
| VL-CDR2 (9B2) | | RTS |
| VL-CDR3 (9B2) | SEQ ID NO: 11 | QQYHSYPLT |
| Mab 9B2 domain VH: | SEQ ID NO: 12 | QVQLQQSGAELAKPGASVKLSCKASGYTFTRYWM |
| FR1-CDR1-FR2-CDR2- | | HWVKQRPGQGLEWIGHINPSSGYTKYNQKFKDKA |
| FR3-CDR3-FR4 | | TLTADKSSSTAYMQLSSLTYEDSAVYYCAREVYY |
| | | YGSSYGDFDYWGQGTTLTVSS |
| Mab 9B2 domain VL: | SEQ ID NO: 13 | QIVLTQSPAIMSASPGEKVTISCSASSSVSYMYW |
| VL FR1-CDR1-FR2- | | YQQKPGSSPKPWIYRTSNLASGVPARFSGSGSGT |
| CDR2-FR3-CDR3-FR4 | | SYSLTISSMEAEDAATYYCQQYHSYPLTFGAGTK |
| | | LELK |

In a first aspect, the invention relates to an isolated anti-β8 chain of αvβ8 integrin (Itgβ8) neutralizing antibody, wherein said antibody specifically binds to β8 chain of αvβ8 integrin expressed on Tregs (i.e β8 chain anchored in Tregs plasmic membrane).

In particular embodiment, the antibodies of the invention specifically bind to β8 chain of αvβ8 integrin expressed on Tregs. According to the invention, the term “β8 chain of αvβ8 integrin expressed on Tregs” refers to the β8 chain exhibiting a specific steric conformation as demonstrated in Lienart S. et al Science 2018, which is hereby incorporated herein by reference.

In particular embodiment, the antibodies of the invention specifically bind to Itgβ expressed on Tregs associated with the tumor microenvironment (TME) (i.e TME-associated Tregs or Tregs in the TME).

In particular embodiment, the antibodies of the invention specifically and only binds to Itgβ expressed on Tregs, and especially to Itgβ expressed on Tregs associated with the TME (i.e TME-associated Tregs or Tregs in the TME).

In particular embodiment, the antibodies of the invention specifically bind to Itgβ expressed on Tregs and does not bind to β8 chain of αvβ8 integrin expressed on other cell types (such as endothelial cells, epithelial cells, fibroblasts, neurons, glial cells, and/or dendritic cells).

As used herein, the term “anti-Itgβ8 neutralizing antibody” has its general meaning in the art and refers to an antibody that can inhibit the biological activity of the Itgβ8, i.e that can block the ability of Tregs to activate TGF-β via αvβ8.

Thus, the invention refers to an isolated anti-Itgβ8 neutralizing antibody wherein said antibody specifically binds to β8 chain of αvβ8 integrin expressed on Tregs and inhibits the ability of Tregs to activate TGF-β via αvβ8.

In another embodiment, the anti-Itgβ8 neutralizing antibody can block the interaction of αvβ8 integrin with TGF-β.

In other words, the anti-Itgβ8 neutralizing antibody is able to restore anti-cancer response of CD8 T cells via inhibition of TGF-β activation and thus TGF-β signaling in CD8 T cells infiltrating the tumor.

Then, for this invention, neutralizing antibodies are selected as above described for their capacity to (i) bind to β8 chain of αvβ8 integrin expressed on Tregs and/or (ii) inhibition of TGF-β activation by Tregs (see example with functional test and FIG. 3), and/or (iii) inhibiting tumor cell growth.

Tests for determining the capacity of an antibody to binds to Itgβ8 expressed on Tregs are well known to the person skilled in the art. In a preferred embodiment, the antagonist specifically binds to Itgβ8 expressed on Tregs in a sufficient manner to inhibit the biological activity of αvβ8 expressed on Tregs, i.e inhibiting the activation of TGF-β. Binding to β8 chain of αvβ8 integrin expressed on Tregs and inhibition of the biological activity of αvβ8 expressed on Tregs may be determined by any competing assays well known in the art. For example, the assay may consist in determining the specific ability of the antibody to bind to β8 chain of αvβ8 integrin expressed on Tregs via flow cytometry (see example with and FIG. 2) for example. Then a competitive assay may be settled to determine the ability of the agent to inhibit biological activity of αvβ8 expressed on Tregs as detailed above.

In particular embodiment, the isolated anti-Itgβ8 neutralizing antibody of the invention is a monoclonal antibody.

In some embodiments, the isolated anti-Itgβ8 neutralizing antibody comprises (a) a heavy chain wherein the variable domain comprises:
a H-CDR1 having the following sequence: G-Y-T-F-T-X₆-Y-X₈ (SEQ ID NO: 14) wherein X₆ is S or R, and X₈ is T or W;
a H-CDR2 having the following sequence: I-N-P-S-S-G-Y-T (SEQ ID NO:2);
a H-CDR3 having the following sequence: A-R-X₃-E-X₅-X₆-X₇-X₈-X₉-X₁₀-X₁₁-Y-X₁₃-X₁₄-X₁₅-X₁₆-X₁₇ (SEQ ID NO:15) wherein X₃ is A or none, X₅ is G or V, X₆ is L or Y, X₇ is R or Y, X₈ is A or Y, X₉ is W or G, X₁₀ is F or S, X₁₁ is A or S, X₁₃ is G or none, X₁₄ is D or none, X₁₅ is F or none, X₁₆ is D or none and X₁₇ is Y or none; and
(b) a light chain wherein the variable domain comprises:
   a L-CDR1 having the following sequence: X₁-X₂-V-X₄-X₅-X₆, wherein X₁ is Q or S, X₂ is N or S, X₄ is G or S, X₅ is T or Y, and X₆ is N or none,
   a L-CDR2 having the following sequence: X₁-X₂-S, wherein X₁ is S or R, and X₂ is A or T,
   a L-CDR3 having the following sequence: Q-Q-Y-X₄-S-Y-P-X₈-T (SEQ ID NO: 16) wherein X₄ is N or H, and X₈ is Y or L.

In some embodiments, the isolated anti-Itgβ8 neutralizing antibody comprises:
(a) a heavy chain wherein the variable domain comprises: a H-CDR1 having a sequence set forth as SEQ ID NO:1 or SEQ ID NO:8; a H-CDR2 having a sequence set forth as SEQ ID NO:2; and a H-CDR3 having a sequence set forth as SEQ ID NO:3 or SEQ ID NO:9; and
(b) a light chain wherein the variable domain comprises: a L-CDR1 having a sequence set forth as SEQ ID NO:4 or SEQ ID NO:10; a L-CDR2 having a sequence set forth as SAS or RTS; and a L-CDR3 having a sequence set forth as SEQ ID NO:5 or SEQ ID NO:11.

In some embodiments, the isolated anti-Itgβ8 neutralizing antibody comprises:
(a) a heavy chain wherein the variable domain comprises: a H-CDR1 having a sequence set forth as SEQ ID NO:1; a H-CDR2 having a sequence set forth as SEQ ID NO:2; and a H-CDR3 having a sequence set forth as SEQ ID NO:3; and
(b) a light chain wherein the variable domain comprises: a L-CDR1 having a sequence set forth as SEQ ID NO:4; a L-CDR2 having a sequence set forth as SAS; and a L-CDR3 having a sequence set forth as SEQ ID NO:5 (“1G10G4 mAb or 1G10H5 mAb”).

In some embodiments, the isolated anti-Itgβ8 neutralizing antibody comprises: (a) a heavy chain wherein the variable domain having at least 70% of identity with a sequence set forth as SEQ ID NO:6, and (b) a light chain wherein the variable domain having at least 70% of identity with a sequence set forth as SEQ ID NO:7 (“1G10G4 mAb or 1G10H5 mAb”).

In some embodiments, the isolated anti-Itgβ8 neutralizing antibody comprises: (a) a heavy chain wherein the variable domain having at least 70% of identity with a sequence set forth as SEQ ID NO:6 and comprises a H-CDR1 having a sequence set forth as SEQ ID NO:1; a H-CDR2 having a sequence set forth as SEQ ID NO:2; and a H-CDR3 having a sequence set forth as SEQ ID NO:3, and (b) a light chain wherein the variable domain having at least 70% of identity with a sequence set forth as SEQ ID NO:7 and comprises a L-CDR1 having a sequence set forth as SEQ ID NO:4; a L-CDR2 having a sequence set forth as SAS; and a L-CDR3 having a sequence set forth as SEQ ID NO:5. (“1G10G4 mAb or 1G10H5 mAb”).

In some embodiments, the isolated anti-Itgβ8 neutralizing antibody comprises or consist in: (a) a heavy chain wherein the variable domain has a sequence set forth as SEQ ID NO:6, and (b) a light chain wherein the variable domain has a sequence set forth as SEQ ID NO:7. (“1G10G4 mAb or 1G10H5 mAb”).

In some embodiments, the isolated anti-Itgβ8 neutralizing antibody comprises:
(a) a heavy chain wherein the variable domain comprises: a H-CDR1 having a sequence set forth as SEQ ID NO:8; a H-CDR2 having a sequence set forth as SEQ ID NO:2; and a H-CDR3 having a sequence set forth as SEQ ID NO:9; and
(b) a light chain wherein the variable domain comprises: a L-CDR1 having a sequence set forth as SEQ ID NO:10; a L-CDR2 having a sequence set forth as RTS; and a L-CDR3 having a sequence set forth as SEQ ID NO:11. (“9B2 mAb”).

In some embodiments, the isolated anti-Itgβ8 neutralizing antibody comprises: (a) a heavy chain wherein the variable domain having at least 70% of identity with a sequence set forth as SEQ ID NO:12, and (b) a light chain wherein the variable domain having at least 70% of identity with a sequence set forth as SEQ ID NO:13. (“9B2 mAb”).

In some embodiments, the isolated anti-Itgβ8 neutralizing antibody comprises: (a) a heavy chain wherein the variable domain having at least 70% of identity with a sequence set forth as SEQ ID NO:12 and comprises a H-CDR1 having a sequence set forth as SEQ ID NO:8; a H-CDR2 having a sequence set forth as SEQ ID NO:2; and a H-CDR3 having a sequence set forth as SEQ ID NO:9, and (b) a light chain wherein the variable domain having at least 70% of identity with a sequence set forth as SEQ ID NO:13 and comprises a L-CDR1 having a sequence set forth as SEQ ID NO:10; a L-CDR2 having a sequence set forth as RTS; and a L-CDR3 having a sequence set forth as SEQ ID NO:11. (“9B2 mAb”).

In some embodiments, the isolated anti-Itgβ8 neutralizing antibody comprises or consist in (a) a heavy chain wherein the variable domain has a sequence set forth as SEQ ID NO:12, and (b) a light chain wherein the variable domain has a sequence set forth as SEQ ID NO:13. (“9B2 mAb”).

The present invention thus provides antibodies comprising functional variants of the VH region including FRs and/or one or more CDRs of the antibodies 1G10H5, 1G10G4 and 9B2.

A functional variant of a VH (FR, or CDR) used in the context of a single domain antibody of the present invention still allows the antibody to retain at least a substantial proportion (at least about 50%, 60%, 70%, 80%, 90%, 95% or more) of the affinity/avidity and/or the specificity/selectivity of the parent antibody (i.e. single domain antibody Z70) and in some cases such a single domain antibody of the present invention may be associated with greater affinity, selectivity and/or specificity than the parent single domain antibody (or VHH). Such variants can be obtained by a number of affinity maturation protocols including mutating the CDRs (Yang et al., J. Mol. Biol., 254, 392-403, 1995), chain shuffling (Marks et al., Bio/Technology, 10, 779-783, 1992), use of mutator strains of E. coli (Low et al., J. Mol. Biol., 250, 359-368, 1996), DNA shuffling (Patten et al., Curr. Opin. Biotechnol., 8, 724-733, 1997), phage display (Thompson et al., J. Mol. Biol., 256, 77-88, 1996) and sexual PCR (Crameri et al., Nature, 391, 288-291, 1998). Vaughan et al. (supra) discusses these methods of affinity maturation. Such functional variants typically retain significant sequence identity to the parent single domain antibody (or VHH). The sequence of CDR variants may differ from the sequence of the CDR of the parent antibody sequences through mostly conservative substitutions; for instance at least about 35%, about 50% or more, about 60% or more, about 70% or more, about 75% or more, about 80% or more, about 85% or more, about 90% or more, (e.g., about 65-95%, such as about 92%, 93% or 94%) of the substitutions in the variant are conservative amino acid residue replacements. The sequences of CDR variants may differ from the sequence of the CDRs of the parent antibody sequences through mostly conservative substitutions; for instance at least 10, such as at least 9, 8, 7, 6, 5, 4, 3, 2 or 1 of the substitutions in the variant are conservative amino acid residue replacements. In the context of the present invention, conservative substitutions may be defined by substitutions within the classes of amino acids reflected as follows:
Aliphatic residues I, L, V, and M
Cycloalkenyl-associated residues F, H, W, and Y
Hydrophobic residues A, C, F, G, H, I, L, M, R, T, V, W, and Y
Negatively charged residues D and E
Polar residues C, D, E, H, K, N, Q, R, S, and T
Positively charged residues H, K, and R
Small residues A, C, D, G, N, P, S, T, and V
Very small residues A, G, and S
Residues involved in turn formation A, C, D, E, G, H, K, N, Q, R, S, P, and T
Flexible residues Q, T, K, S, G, P, D, E, and R
More conservative substitutions groupings include: valine-leucine-isoleucine, phenylalanine-tyrosine, lysine-arginine, alanine-valine, and asparagine-glutamine. Conservation in terms of hydropathic/hydrophilic properties and residue weight/size also is substantially retained in a variant CDR as compared to a CDR of Z70. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The retention of similar residues may also or alternatively be measured by a similarity score, as determined by use of a BLAST program (e.g., BLAST 2.2.8 available through the NCBI using standard settings BLOSUM62, Open Gap=1 and Gap extension=1).

In some embodiments, the isolated anti-Itgβ8 neutralizing antibody of the invention is a “humanized” antibody.

According to the invention, the term “humanized antibody” refers to an antibody having variable region framework and constant regions from a human antibody but retains the CDRs of a previous non-human antibody.

The humanized antibody of the present invention may be produced by obtaining nucleic acid sequences encoding CDR domains, as previously described, constructing a humanized antibody expression vector by inserting them into an expression vector for animal cell having genes encoding (i) a heavy chain constant region identical to that of a human antibody and (ii) a light chain constant region identical to that of a human antibody, and expressing the genes by introducing the expression vector into an animal cell. The humanized antibody expression vector may be either of a type in which a gene encoding an antibody heavy chain and a gene encoding an antibody light chain exists on separate vectors or of a type in which both genes exist on the same vector (tandem type). In respect of easiness of construction of a humanized antibody expression vector, easiness of introduction into animal cells, and balance between the expression levels of antibody H and L chains in animal cells, humanized antibody expression vector of the tandem type is preferred. Examples of tandem type humanized antibody expression vector include pKANTEX93 (WO 97/10354), pEE18 and the like. Methods for producing humanized antibodies based on conventional recombinant DNA and gene transfection techniques are well known in the art (See, e. g., Riechmann L. et al. 1988; Neuberger M S. et al. 1985). Antibodies can be humanized using a variety of techniques known in the art including, for example, CDR-grafting (EP 239,400; PCT publication WO91/09967; U.S. Pat. Nos. 5,225,539; 5,530,101; and 5,585,089), veneering or resurfacing (EP 592,106; EP 519,596; Padlan E A (1991); Studnicka G M et al. (1994); Roguska M A. et al. (1994)), and chain shuffling (U.S. Pat. No. 5,565,332). The general recombinant DNA technology for preparation of such antibodies is also known (see European Patent Application EP 125023 and International Patent Application WO 96/02576).

In one embodiment, the isolated anti-Itgβ8 neutralizing antibody of the invention is a chimeric antibody, particularly a chimeric mouse/human antibody.

According to the invention, the term “chimeric antibody” refers to an antibody which comprises a VH domain and a VL domain of a non-human antibody, and a CH domain and a CL domain of a human antibody.

In some embodiments, the chimeric antibody of the present invention can be produced by obtaining nucleic sequences encoding VL and VH domains as previously described, constructing a human chimeric antibody expression vector by inserting them into an expression vector for animal cell having genes encoding human antibody CH and human antibody CL, and expressing the coding sequence by introducing the expression vector into an animal cell. As the CH domain of a human chimeric antibody, it may be any region which belongs to human immunoglobulin, but those of IgG class are suitable and any one of subclasses belonging to IgG class, such as IgG1, IgG2, IgG3 and IgG4, can also be used. Also, as the CL of a human chimeric antibody, it may be any region which belongs to Ig, and those of kappa class or lambda class can be used. Methods for producing chimeric antibodies involve conventional recombinant DNA and gene transfection techniques are well known in the art (See Morrison S L. et al. (1984) and patent documents U.S. Pat. Nos. 5,202,238; and 5,204,244).

In one embodiment, the isolated anti-Itgβ8 neutralizing antibody of the invention is an antigen biding fragment selected from the group consisting of a Fab, a F(ab)′2, a single domain antibody, a ScFv, a Sc(Fv)2, a diabody, a triabody, a tetrabody, an unibody, a minibody, a maxibody, a small modular immunopharmaceutical (SMIP), minimal recognition units consisting of the amino acid residues that mimic the hypervariable region of an antibody as an isolated complementary determining region (CDR), and fragments which comprise or consist of the VL as well as amino acid sequence having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with SEQ ID NO:7 or SEQ ID NO:13 and/or VH chains as well as amino acid sequence having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with SEQ ID NO:6 or SEQ ID NO:12.

In one embodiment, the isolated anti-Itgβ8 neutralizing antibody of the invention is an antigen biding fragment which comprise or consist of the VL as well as amino acid sequence having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with SEQ ID NO:7 and/or VH chains as well as amino acid sequence having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with SEQ ID NO:6.

In one embodiment, the isolated anti-Itgβ8 neutralizing antibody of the invention is an antigen biding fragment which comprise or consist of the VL as well as amino acid sequence having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with SEQ ID NO:12 and/or VH chains as well as amino acid sequence having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with SEQ ID NO:13.

The term “antigen binding fragment” of an antibody, as used herein, refers to one or more fragments of an intact antibody that retain the ability to specifically binds to a given antigen (e.g. β8 chain of αvβ8 integrin expressed on T regulatory cells). Antigen biding functions of an antibody can be performed by fragments of an intact antibody. Examples of biding fragments encompassed within the term antigen biding fragment of an antibody include a Fab fragment, a monovalent fragment consisting of the VL, VH, CL and CH1 domains; a Fab′ fragment, a monovalent fragment consisting of the VL, VH, CL, CH1 domains and hinge region; a F(ab′)₂ fragment, a bivalent fragment comprising two Fab′ fragments linked by a disulfide bridge at the hinge region; an Fd fragment consisting of VH domains of a single arm of an antibody; a single domain antibody (sdAb) fragment (Ward et al., 1989 Nature 341:544-546), which consists of a VH domain or a VL domain; and an isolated complementary determining region (CDR). Furthermore, although the two domains of the Fv fragment, VL and VH, are coded for by separate genes, they can be joined, using recombinant methods, by an artificial peptide linker that enables them to be made as a single protein chain in which the VL and VH regions pair to form monovalent molecules (known as single chain Fv (ScFv); see, e.g., Bird et al., 1989 Science 242:423-426; and Huston et al., 1988 proc. Natl. Acad. Sci. 85:5879-5883). “dsFv” is a VH::VL heterodimer stabilised by a disulfide bond. Divalent and multivalent antibody fragments can form either spontaneously by association of monovalent scFvs, or can be generated by coupling monovalent scFvs by a peptide linker, such as divalent sc(Fv)2. Such single chain antibodies include one or more antigen biding portions or fragments of an antibody. These antibody fragments are obtained using conventional techniques known to those skilled in the art, and the fragments are screened for utility in the same manner as are intact antibodies. A unibody is another type of antibody fragment lacking the hinge region of IgG4 antibodies. The deletion of the hinge region results in a molecule that is essentially half the size of traditional IgG4 antibodies and has a univalent binding region rather than the bivalent biding region of IgG4 antibodies. Antigen binding fragments can be incorporated into single domain antibodies, SMIP, maxibodies, minibodies, intrabodies, diabodies, triabodies and tetrabodies (see, e.g., Hollinger and Hudson, 2005, Nature Biotechnology, 23, 9, 1126-1136). The term “diabodies” “tribodies” or “tetrabodies” refers to small antibody fragments with multivalent antigen-binding sites (2, 3 or four), which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Antigen biding fragments can be incorporated into single chain molecules comprising a pair of tandem Fv segments (VH-CH1-VH-CH1) Which, together with complementary light chain polypeptides, form a pair of antigen binding regions (Zapata et al., 1995 Protein Eng. 8(10); 1057-1062 and U.S. Pat. No. 5,641,870).

The Fab of the present invention can be obtained by treating the antibody which specifically reacts with the β8 chain of αvβ8 integrin expressed on T regulatory cells according to the invention with a protease, papaine. Also, the Fab can be produced by inserting DNA encoding Fab of the antibody into a vector for prokaryotic expression system, or for eukaryotic expression system, and introducing the vector into a procaryote or eucaryote (as appropriate) to express the Fab.

The F(ab′)2 of the present invention can be obtained treating the antibody which specifically reacts with the β8 chain of αvβ8 integrin expressed on T regulatory cells according to the invention with a protease, pepsin. Also, the F(ab′)2 can be produced by binding Fab′ described below via a thioether bond or a disulfide bond.

The Fab′ of the present invention can be obtained treating the F(ab′)2 which specifically reacts with β8 chain of αvβ8 integrin expressed on T regulatory cells according to the invention with a reducing agent, dithiothreitol. Also, the Fab′ can be produced by inserting DNA encoding Fab′ fragment of the antibody into an expression vector for prokaryote, or an expression vector for eukaryote, and introducing the vector into a prokaryote or eukaryote (as appropriate) to perform its expression.

The scFv of the present invention can be produced by obtaining cDNA encoding the VH and VL domains as previously described, constructing DNA encoding scFv, inserting the DNA into an expression vector for prokaryote, or an expression vector for eukaryote, and then introducing the expression vector into a prokaryote or eukaryote (as appropriate) to express the scFv. To generate a humanized scFv fragment, a well-known technology called CDR grafting may be used, which involves selecting the complementary determining regions (CDRs) from a donor scFv fragment, and grafting them onto a human scFv fragment framework of known three dimensional structure (see, e. g., WO98/45322; WO 87/02671; U.S. Pat. Nos. 5,859,205; 5,585,089; 4,816,567; EP0173494).

Domain Antibodies (dAbs) are the smallest functional binding units of antibodies—molecular weight approximately 13 kDa—and correspond to the variable regions of either the heavy (VH) or light (VL) chains of antibodies. Further details on domain antibodies and methods of their production are found in U.S. Pat. Nos. 6,291,158; 6,582,915; 6,593,081; 6,172,197; and 6,696,245; US 2004/0110941; EP 1433846, 0368684 and 0616640; WO 2005/035572, 2004/101790, 2004/081026, 2004/058821, 2004/003019 and 2003/002609, each of which is herein incorporated by reference in its entirety.

UniBodies are another antibody fragment technology, based upon the removal of the hinge region of IgG4 antibodies. The deletion of the hinge region results in a molecule that is essentially half the size of a traditional IgG4 antibody and has a univalent binding region rather than a bivalent binding region. Furthermore, because UniBodies are about smaller, they may show better distribution over larger solid tumors with potentially advantageous efficacy. Further details on UniBodies may be obtained by reference to WO 2007/059782, which is incorporated by reference in its entirety.

The isolated anti-Itgβ8 neutralizing antibody of the present invention may be of any isotype. The choice of isotype typically will be guided by the desired effector functions. IgG1 and IgG3 are isotypes that mediate such effectors functions as ADCC or CDC, when IgG2 and IgG4 don't or in a lower manner. Either of the human light chain constant regions, kappa or lambda, may be used. If desired, the class of a monoclonal antibody of the present invention may be switched by known methods. Typical, class switching techniques may be used to convert one IgG subclass to another, for instance from IgG1 to IgG2. Thus, the effector function of the monoclonal antibodies of the present invention may be changed by isotype switching to, e.g., an IgG1, IgG2, IgG3, IgG4, IgD, IgA, IgE, or IgM antibody for various therapeutic uses.

In some embodiments, the isolated anti-Itgβ8 neutralizing antibody is an IgM antibody.

In some embodiments, the isolated anti-Itgβ8 neutralizing antibody is an IgG1, IgG2, IgG3 or IgG4 antibody.

A further aspect of the invention refers to a cross-competing antibody which cross-competes for binding the β8 chain of αvβ8 integrin expressed on T regulatory cells with the antibody of the invention.

In some embodiment, the cross-competing single-domain antibody of the present invention cross-competes for binding the β8 chain of αvβ8 integrin expressed on T regulatory cells with the antibody comprising a heavy chain having a sequence set forth as SEQ ID NO:6, and (b) a light chain having a sequence set forth as SEQ ID NO:7. (“1G10G4 or 1G10H5 mAb”).

In some embodiment, the cross-competing single-domain antibody of the present invention cross-competes for binding the β8 chain of αvβ8 integrin expressed on T regulatory cells with the antibody comprising a heavy chain having a sequence set forth as SEQ ID NO:12, and (b) a light chain having a sequence set forth as SEQ ID NO:13. (“9B2 mAb”).

As used herein, the term “cross-competes” refers to single-domain antibodies which share the ability to bind to a specific region of an antigen. In the present disclosure the single-domain antibody that “cross-competes” has the ability to interfere with the binding of another single-domain antibody for the antigen in a standard competitive binding assay. Such a single-domain antibody may, according to non-limiting theory, bind to the same or a related or nearby (e.g., a structurally similar or spatially proximal) epitope as the single-domain antibody with which it competes. Cross-competition is present if single-domain antibody A reduces binding of single-domain antibody B at least by 60%, specifically at least by 70% and more specifically at least by 80% and vice versa in comparison to the positive control which lacks one of said single-domain antibodies. As the skilled artisan appreciates competition may be assessed in different assay set-ups. One suitable assay involves the use of the Flow cytometry (using Flow cytometers and fluorescently-labeled cell suspension). Another possible assay for measuring cross-competition uses an cell-based ELISA approach.

According to the present invention, the cross-competing antibody as above described retain the activity of the antibody of the invention (i.e able to inhibit the ability of Tregs to activate TGF-β via αvβ8).

In some embodiments, the cross-competing antibody as above described retain the activity of the antibody comprising or consisting a heavy chain having a sequence set forth as SEQ ID NO:6, and (b) a light chain having a sequence set forth as SEQ ID NO:7. (“1G10G4 or 1G10H5 mAb”).

In some embodiments, the cross-competing antibody as above described retain the activity of the antibody comprising or consisting a heavy chain having a sequence set forth as SEQ ID NO:12, and (b) a light chain having a sequence set forth as SEQ ID NO:13. (“9B2 mAb”).

Thus, in some embodiment, the cross-competing antibody of the present invention is anti-Itgβ8 neutralizing antibody, wherein said cross-competing antibody specifically binds to β8 chain of αvβ8 integrin expressed on Tregs and is able to inhibit the ability of Tref to activate TGF-β.

In some embodiments, the isolated anti-Itgβ8 neutralizing antibody is engineered in order to improve its properties.

Engineered antibodies of the invention include those in which modifications have been made to framework residues within VH and/or VL, e.g. to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis. Such “backmutated” antibodies are also intended to be encompassed by the invention. Another type of framework modification involves mutating one or more residues within the framework region, or even within one or more CDR regions, to remove T cell—epitopes to thereby reduce the potential immunogenicity of the antibody. This approach is also referred to as “deimmunization” and is described in further detail in U.S. Patent Publication No. 20030153043 by Carr et al.

In some embodiments, the glycosylation of the isolated anti-Itgβ8 neutralizing antibody of the invention is modified. Glycosylation can be altered to, for example, increase the affinity of the antibody for the antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

In another embodiment, the isolated anti-Itgβ8 neutralizing antibody of the invention is modified to increase its biological half-life. Various approaches are possible. For example, one or more of the following mutations can be introduced: T252L, T254S, T256F, as described in U.S. Pat. No. 6,277,375 by Ward. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. Antibodies with increased half-lives and improved binding to the neonatal Fc receptor (FcRn), which is responsible for the transfer of maternal IgGs to the foetus (Guyer et al., J. Immunol. 117:587 (1976) and Kim et al., J. immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311,312, 317, 340, 356, 360, 362, 376, 378, 380, 382, 413, 424, or 434, e.g., substitutions of Fc region residue 434 (U.S. Pat. No. 7,371,826).

Another modification of the antibodies the invention herein that is contemplated by the invention is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See for example, EP0154316 by Nishimura et al. and EP0401384 by Ishikawa et al.

Another modification of the antibodies of the invention that is contemplated by the invention is a conjugate or a protein fusion of at least the antigen-binding region of the antibody of the invention to serum protein, such as human serum albumin or a fragment thereof to increase half-life of the resulting molecule. Such approach is for example described in Ballance et al. EP0322094. Another possibility is a fusion of at least the antigen-binding region of the antibody of the invention to proteins capable of binding to serum proteins, such human serum albumin to increase half-life of the resulting molecule. Such approach is for example described in Nygren et al., EP 0 486 525.

Polysialytion is another technology, which uses the natural polymer polysialic acid (PSA) to prolong the active life and improve the stability of therapeutic peptides and proteins. PSA is a polymer of sialic acid (a sugar). When used for protein and therapeutic peptide drug delivery, polysialic acid provides a protective microenvironment on conjugation. This increases the active life of the therapeutic protein in the circulation and prevents it from being recognized by the immune system. The PSA polymer is naturally found in the human body. It was adopted by certain bacteria which evolved over millions of years to coat their walls with it. These naturally polysialylated bacteria were then able, by virtue of molecular mimicry, to foil the body's defense system. PSA, nature's ultimate stealth technology, can be easily produced from such bacteria in large quantities and with predetermined physical characteristics. Bacterial PSA is completely non-immunogenic, even when coupled to proteins, as it is chemically identical to PSA in the human body.

Another technology includes the use of hydroxyethyl starch (“HES”) derivatives linked to antibodies. HES is a modified natural polymer derived from waxy maize starch and can be metabolized by the body's enzymes. HES solutions are usually administered to substitute deficient blood volume and to improve the rheological properties of the blood. Hesylation of an antibody enables the prolongation of the circulation half-life by increasing the stability of the molecule, as well as by reducing renal clearance, resulting in an increased biological activity. By varying different parameters, such as the molecular weight of HES, a wide range of HES antibody conjugates can be customized.

The antibodies of the present invention are produced by any technique known in the art, such as, without limitation, any chemical, biological, genetic or enzymatic technique, either alone or in combination. Typically, knowing the amino acid sequence of the desired sequence, one skilled in the art can readily produce said antibodies, by standard techniques for production of polypeptides. For instance, they can be synthesized using well-known solid phase method, preferably using a commercially available peptide synthesis apparatus (such as that made by Applied Biosystems, Foster City, California) and following the manufacturer's instructions. Alternatively, antibodies of the present invention can be synthesized by recombinant DNA techniques well-known in the art. For example, antibodies can be obtained as DNA expression products after incorporation of DNA sequences encoding the antibodies into expression vectors and introduction of such vectors into suitable eukaryotic or prokaryotic hosts that will express the desired antibodies, from which they can be later isolated using well-known techniques.

In particular embodiment, the anti-Itgβ8 neutralizing antibody of the invention has been produced by in vitro immunization approach (or hybridoma technology).

As used herein, the “in vitro immunization approach” or “hybridoma technology” has its general meaning in the art and refers to immunization of host animals, such as mice, with the antigen of interest (or immunogen agent), such as according to the invention Tregs expressing αvβ8 in order to initiate an appropriate and specific immune response. Normally, the mammalian immune system has built up a highly specific immune response after 3 months. Then the B cells are harvested and are fused with immortal B cancer cells, a myeloma to produce a hybrid cell line called a hybridoma. The hybridoma is then grown in culture, each culture starting with one viable hybridoma cell which produces one monoclonal antibody per culture.

In particular embodiment, the anti-Itgβ8 neutralizing antibody of the invention has been produced by in vitro immunization approach (or hybridoma technology) by using Tregs expressing αvβ8 as immunogen agent.

### 2) Nucleic Acids, Vectors, Recombinant Host Cells

A further object of the invention relates to a nucleic acid molecule encoding an anti-Itgβ8 neutralizing antibody according to the invention. More particularly the nucleic acid molecule encodes a heavy chain and/or a light chain of an anti-Itgβ8 neutralizing antibody of the present invention.

In particular embodiment, the nucleic acid comprises a nucleic acid sequence having 70% of identity with SEQ ID NO:17 and/or SEQ ID NO:18.

In particular embodiment, the nucleic acid comprises a nucleic acid sequence set forth as SEQ ID NO:17 and/or SEQ ID NO:18.

Heavy chain variable acid nucleic sequence
1G10G4 or 1G10H5
SEQ ID NO: 17>
CAGGTCCAGCTGCAGCAGTCTGGGGCTGAACTGGCAAGACCTGGTGCCTC
AGTGAAGATGTCCTGCAAGGCTTCTGGCTACACCTTTACTAGCTACACGA
TGCACTGGGTAAAACAGAGGCCTGGACAGGGTCTGGAATGGATTGGATAC
ATTAATCCTAGCAGTGGTTATACTAAGTACAATCAGAAGTTCAAGGACAA
GGCCACATTGACTGCAGACAAATCCTCCAGCACAGCCTACATGCAACTGA
GCAGCCTGACATCTGAGGACTCTGCAGTCTATTACTGTGCAAGAGCTGAG
GGATTAAGGGCCTGGTTTGCTTACTGGGGCCAAGGGACTCTGGTCACTGT
CTCTGCA
Light chain variable acid nucleic sequence
1G10G4 or 1G10H5
SEQ ID NO: 18>
GACATTGTGATGACCCAGTCTCAAAAATTCATGTCCACATCAGTAGGAGA
CAGGGTCAGCGTCACCTGCAAGGCCAGTCAGAATGTGGGTACTAATGTAG
CCTGGTATCAGCAGAAACCAGGGCAATCTCCTAAAGCACTGATTTACTCG
GCATCCTACCGGTACAGTGGAGTCCCTGATCACTTCACAGGCAGTGGATC
TGGGACAGATTTCACTCTCACCATCATCAATGTGCAGTCTGAAGACTTGG
CAGAGTATTTCTGTCAGCAATATAACAGCTATCCGTACACGTTCGGAGGG
GGGACCAAGCTGGAAATAAAA

In particular embodiment, the nucleic acid comprises a nucleic acid sequence having 70% of identity with SEQ ID NO:19 and/or SEQ ID NO:20.

In particular embodiment, the nucleic acid comprises a nucleic acid sequence set forth as SEQ ID NO:19 and/or SEQ ID NO:20.

Heavy chain variable acid nucleic sequence
9B2
SEQ ID NO: 19>
CAGGTCCAGCTGCAGCAGTCTGGGGCTGAACTGGCAAAACCTGGGGCCTC
AGTGAAGCTGTCCTGCAAGGCTTCTGGCTACACCTTTACTAGGTACTGGA
TGCACTGGGTAAAACAGAGGCCTGGACAGGGTCTGGAATGGATTGGACAC
ATTAATCCTAGCAGTGGTTATACTAAGTACAATCAGAAGTTCAAGGACAA
GGCCACATTGACTGCAGACAAATCCTCCAGCACAGCCTACATGCAGCTGA
GCAGCCTGACATATGAGGACTCTGCAGTCTATTACTGTGCAAGAGAGGTT
TATTACTACGGTAGTAGCTACGGAGACTTTGACTACTGGGGCCAAGGCAC
CACTCTCACAGTCTCCTCA
Light chain variable acid nucleic sequence
9B2
SEQ ID NO: 20>
CAAATTGTTCTCACCCAGTCTCCAGCAATCATGTCTGCATCTCCAGGGGA
GAAGGTCACCATATCCTGCAGTGCCAGCTCAAGTGTAAGTTACATGTACT
GGTACCAGCAGAAGCCAGGATCCTCCCCCAAACCCTGGATTTATCGCACA
TCCAACCTGGCTTCTGGAGTCCCTGCTCGCTTCAGTGGCAGTGGGTCTGG
GACCTCTTACTCTCTCACAATCAGCAGCATGGAGGCTGAAGATGCTGCCA
CTTATTACTGCCAGCAGTATCATAGTTACCCACTCACGTTCGGTGCTGGG
ACCAAGCTGGAGCTGAAA

Typically, said nucleic acid is a DNA or RNA molecule, which may be included in any suitable vector, such as a plasmid, cosmid, episome, artificial chromosome, phage or a viral vector. As used herein, the terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. The terms “expression vector,” “expression construct” or “expression cassette” are used interchangeably throughout this specification and are meant to include any type of genetic construct containing a nucleic acid coding for a gene product in which part or all of the nucleic acid encoding sequence is capable of being transcribed.

So, a further aspect of the invention relates to a vector comprising a nucleic acid of the invention. Such vectors may comprise regulatory elements, such as a promoter, enhancer, terminator and the like, to cause or direct expression of said antibody upon administration to a subject. Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like. Any expression vector for animal cell can be used, so long as a gene encoding the human antibody C region can be inserted and expressed. Examples of suitable vectors include pAGE107 (Miyaji H et al. 1990), pAGE103 (Mizukami T et al. 1987), pHSG274 (Brady G et al. 1984), pKCR (O'Hare K et al. 1981), pSG1 beta d2-4-(Miyaji H et al. 1990) and the like. Other examples of plasmids include replicating plasmids comprising an origin of replication, or integrative plasmids, such as for instance pUC, pcDNA, pBR, and the like. Other examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv⁺ cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

The choice of a suitable expression vector for expression of the peptides or polypeptides of the invention will of course depend upon the specific host cell to be used, and is within the skill of the ordinary artisan.

Expression requires that appropriate signals be provided in the vectors, such as enhancers/promoters from both viral and mammalian sources that may be used to drive expression of the nucleic acids of interest in host cells. Usually, the nucleic acid being expressed is under transcriptional control of a promoter. A “promoter” refers to a DNA sequence recognized by the synthetic machinery of the cell, or introduced synthetic machinery, required to initiate the specific transcription of a gene. Nucleotide sequences are operably linked when the regulatory sequence functionally relates to the DNA encoding the protein of interest (e.g., a single domain antibody). Thus, a promoter nucleotide sequence is operably linked to a given DNA sequence if the promoter nucleotide sequence directs the transcription of the sequence.

A further aspect of the invention relates to a host cell which has been transfected, infected or transformed by a nucleic acid and/or a vector according to the invention.

The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA bas been “transformed”.

The nucleic acids of the invention may be used to produce an antibody of the present invention in a suitable expression system. The term “expression system” means a host cell and compatible vector under suitable conditions, e.g. for the expression of a protein coded for by foreign DNA carried by the vector and introduced to the host cell. Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al; 1980), rat YB2/3HL.P2.G11.16Ag.20 cell (ATCC CRL1662, hereinafter referred to as “YB2/0 cell”), and the like. The present invention also relates to a method of producing a recombinant host cell expressing an antibody according to the invention, said method comprising the steps of: (i) introducing in vitro or ex vivo a recombinant nucleic acid or a vector as described above into a competent host cell, (ii) culturing in vitro or ex vivo the recombinant host cell obtained and (iii), optionally, selecting the cells which express and/or secrete said antibody. Such recombinant host cells can be used for the production of antibodies of the present invention.

Antibodies of the present invention are suitably separated from the culture medium by conventional immunoglobulin purification procedures such as, for example, protein A-Sepharose, hydroxylapatite chromatography, gel electrophoresis, dialysis, or affinity chromatography.

### 3) Therapeutic Methods and Uses

The inventors previously demonstrated that β8 chain of αvβ8 integrin (Itgβ8) expressed on Tregs specifically promotes TGF-β activation and impairs the CD8 T cell anti-cancer response within the TME of different cancers in both mice and humans (Lainé et al, 2021). Thus, the anti-Itgβ8 neutralizing antibodies of the invention are particularly suitable to restore anti-cancer response of CD8 T cells and to treat cancer.

Accordingly, in some embodiments, the anti-Itgβ8 neutralizing antibody of the invention or a fragment thereof directly binds to β8 chain of αvβ8 expressed on Tregs and inhibit the ability of Tregs to activate TGF-β activation.

In some embodiments, the anti-Itgβ8 neutralizing antibody of the invention or a fragment thereof directly binds to β8 chain of αvβ8 integrin expressed on Tregs and restore anti-tumoral CD8 T cells response (or inhibits the inhibition of CD8 T cell activation via TGF-β).

In some embodiments, the anti-Itgβ8 neutralizing antibody of the invention or a fragment thereof directly binds to β8 chain of αvβ8 integrin expressed on Tregs and restore anti-tumoral CD8 T cells response (or inhibits the inhibition of CD8 T cell activation via TGF-β) in TME.

Thus, the present invention also relates to an anti-Itgβ8 neutralizing antibody of the invention or a fragment thereof for use in a method to activate the anti-tumoral CD8 T cells response of a subject affected with a cancer.

As used herein, the terms “anti-tumoral CD8 T cells response” has its general meaning in the art and refers to the natural ability of the CD8 T cells to lyse cancer cells (as disclosed in Robbins and Kawakami; 1996, Romero, 1996).

As used herein, the term “subject” denotes a mammal, such as a rodent, a feline, a canine, and a primate. Preferably, a subject according to the invention is a human. Preferably, a subject according to the invention is a human afflicted with or susceptible to be afflicted with cancer.

In another aspect, the invention refers to a method for treating cancer in a subject in need thereof, comprising administering comprising administering to said subject an effective amount of the anti-Itgβ8 neutralizing antibody of the invention or a fragment thereof.

As used herein, the term “cancer” refers to an abnormal cell having the capacity for autonomous growth, i.e., an abnormal state or condition characterized by rapidly proliferating cell growth with the potential to invade or spread to other parts of the body. The term is meant to include all types of cancerous growths or oncogenic processes, metastatic tissues or malignantly transformed cells, tissues, or organs, irrespective of histopathologic type or stage of invasiveness. The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, glioblastoma non-small cell carcinoma of the lung, cancer of the small intestine and cancer of the esophagus.

As used herein, the term “cancer” has its general meaning in the art and includes, but is not limited to, solid tumors and blood borne tumors. The term cancer includes diseases of the skin, tissues, organs, bone, cartilage, blood and vessels. The term “cancer” further encompasses both primary and metastatic cancers. Examples of cancers include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus.

In particular embodiment, the cancer is selected from the group consisting of, but not limited to, head and neck squamous cell carcinoma (HNSCC); adrenal cortical cancer; anal cancer; periphilar cancer; distal bile duct cancer; intrahepatic bile duct cancer; osteoblastoma; osteochrondroma; hemangioma; chondromyxoid fibroma; astrocytoma; ductal carcinoma in situ; gynecomastia; endometrial adenocarcinoma; adenocanthoma; papillary serous adenocarcinoma; laryngeal and hypopharyngeal cancer; hemangioma, hepatic adenoma; focal nodular hyperplasia; small cell lung cancer; non-small cell lung cancer; mesothelioma, plasmacytoma; esthesioneuroblastoma; midline granuloma; nasopharyngeal cancer; oral cavity and oropharyngeal cancer, ovarian cancer; pancreatic cancer; penile cancer; pituitary cancer; prostate cancer; salivary gland cancer; non-melanoma skin cancer; stomach cancer, testicular cancer; thymus cancer; follicular carcinoma; anaplastic carcinoma; poorly differentiated carcinoma; medullary thyroid carcinoma; vaginal cancer, vulvar cancer, uterine leiomyosarcoma; bladderneoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating ductal carcinoma; medullary carcinoma; infiltrating lobular carcinoma; lobular carcinoma in situ; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; gliomas; medulloblastoma; Schwannoma; germinoma; craniopharyngioma; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; chorioadenoma destruens; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; sezary syndrome malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the cancer is a solid cancer.

In preferred embodiments, the cancer is selected from the group consisting of glioma, thyroid cancer, colorectal cancer, head and neck cancer, stomach cancer, liver cancer, pancreatic cancer, renal cancer, ovarian cancer, laryngeal squamous cell carcinoma, lung cancer, prostate cancer, cervical cancer, testis cancer, endometrial cancer, breast cancer and melanoma cancer.

In more preferred embodiments, the cancer is breast cancer or melanoma cancer.

In some embodiment, the anti-Itgβ8 neutralizing antibody of the invention or a fragment thereof can be administered in combination with anti-cancer therapy.

As used herein, the term “anti-cancer therapy” has its general meaning in the art and refers to any compound, natural or synthetic, used for the treatment of cancer.

In a particular embodiment, the classical treatment refers to radiation therapy, antibody therapy or chemotherapy.

As used herein, the term “chemotherapeutic agent” refers to chemical compounds that are effective in inhibiting tumor growth. Examples of chemotherapeutic agents include multkinase inhibitors such as sorafenib and sunitinib, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33: 183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalamycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defo famine; demecolcine; diaziquone; elfomithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisp latin and carbop latin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylomithine (DMFO); retinoic acid; capecitabine; and pharmaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

As used herein, the term “radiation therapy” has its general meaning in the art and refers the treatment of cancer with ionizing radiation. Ionizing radiation deposits energy that injures or destroys cells in the area being treated (the target tissue) by damaging their genetic material, making it impossible for these cells to continue to grow. One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons produce x-rays of increasingly greater energy. The use of machines to focus radiation (such as x-rays) on a cancer site is called external beam radiation therapy. Gamma rays are another form of photons used in radiation therapy. Gamma rays are produced spontaneously as certain elements (such as radium, uranium, and cobalt 60) release radiation as they decompose, or decay. In some embodiments, the radiation therapy is external radiation therapy. Examples of external radiation therapy include, but are not limited to, conventional external beam radiation therapy; three-dimensional conformal radiation therapy (3D-CRT), which delivers shaped beams to closely fit the shape of a tumor from different directions; intensity modulated radiation therapy (IMRT), e.g., helical tomotherapy, which shapes the radiation beams to closely fit the shape of a tumor and also alters the radiation dose according to the shape of the tumor; conformal proton beam radiation therapy; image-guided radiation therapy (IGRT), which combines scanning and radiation technologies to provide real time images of a tumor to guide the radiation treatment; intraoperative radiation therapy (IORT), which delivers radiation directly to a tumor during surgery; stereotactic radiosurgery, which delivers a large, precise radiation dose to a small tumor area in a single session; hyperfractionated radiation therapy, e.g., continuous hyperfractionated accelerated radiation therapy (CHART), in which more than one treatment (fraction) of radiation therapy are given to a subject per day; and hypofractionated radiation therapy, in which larger doses of radiation therapy per fraction is given but fewer fractions.

As used herein, the term “immune checkpoint inhibitor” refers to molecules that totally or partially reduce, inhibit, interfere with or modulate one or more immune checkpoint proteins.

As used herein, the term “immune checkpoint protein” has its general meaning in the art and refers to a molecule that is expressed by T lymphocytes in that either turn up a signal (stimulatory checkpoint molecules) or turn down a signal (inhibitory checkpoint molecules).

Examples of stimulatory checkpoint include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, PD-L1, LAG-3, TIM-3 and VISTA.

The compounds used in connection with the treatment methods of the present invention are administered and dosed in accordance with good medical practice, taking into account the clinical condition of the individual subject, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including, but not limited to, improved survival rate or more rapid recovery, or improvement or elimination of symptoms and other indicators as are selected as appropriate measures by those skilled in the art.

### 4) Pharmaceutical Compositions of the Invention

The anti-Itgβ8 neutralizing antibody of the invention or a fragment thereof as described above may be combined with pharmaceutically acceptable excipients, and optionally sustained-release matrices, such as biodegradable polymers, to form therapeutic compositions.

Accordingly, the present invention relates to a pharmaceutical composition comprising i) an anti-Itgβ8 neutralizing antibody according to the invention or a fragment thereof and ii) a pharmaceutically acceptable carrier.

The present invention also relates to a pharmaceutical composition for use in the prevention or treatment of cancer comprising i) an anti-Itgβ8 neutralizing antibody according to the invention or a fragment thereof and ii) a pharmaceutically acceptable carrier.

In therapeutic applications, compositions are administered to a patient already suffering from a disease, as described, in an amount sufficient to cure or at least partially stop the symptoms of the disease and its complications. An appropriate dosage of the pharmaceutical composition is readily determined according to any one of several well-established protocols. For example, animal studies (for example on mice or rats) are commonly used to determine the maximal tolerable dose of the bioactive agent per kilogram of weight. In general, at least one of the animal species tested is mammalian. The results from the animal studies can be extrapolated to determine doses for use in other species, such as humans for example. What constitutes an effective dose also depends on the nature and severity of the disease or condition, and on the general state of the patient's health.

In therapeutic treatments, the antagonist contained in the pharmaceutical composition can be administered in several dosages or as a single dose until a desired response has been achieved. The treatment is typically monitored and repeated dosages can be administered as necessary.

The daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 10 mg/kg of body weight per day. It will be understood, however, that the specific dose level and frequency of dosage for any particular patient may be varied and will depend upon a variety of factors including the activity of the specific compound employed, the metabolic stability, and length of action of that compound, the age, the body weight, general health, sex, diet, mode and time of administration, rate of excretion, drug combination, the severity of the particular condition, and the host undergoing therapy.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

The appropriate unit forms of administration include forms for oral administration, such as tablets, gelatine capsules, powders, granules and solutions or suspensions to be taken orally, forms for sublingual and buccal administration, aerosols, implants, forms for subcutaneous, intramuscular, intravenous, intranasal or intraocular administration and forms for rectal administration.

The invention will be further illustrated by the following figures and examples. However, these examples and figures should not be interpreted in any way as limiting the scope of the present invention.

### FIGURES

FIG. 1: Itgβ8 is largely expressed on Tregs in the TME in both mice and humans.

(A-F) Itgβ8-td-Tomato reporter mice were injected with melanoma cells (B16) or breast cancer cells (E0771) in the dermis or in the mammary gland respectively. 18 days later tumors were analyzed by flow cytometry. Percentages of the gated populations are mentioned on dot plots and counter plots. A) Representative plots illustrate the Itgβ8-tdTomato expression in tumors. B) Histograms demonstrating the percentages of Itgβ8^{pos} cells among the hematopoietic compartment (CD45^{pos}) and the non-hematopoietic compartment (CD45^{neg}) in 5 tumors of each. C, E) Representative contour plots illustrating the proportion of CD3^{pos} cells among Itgβ8-td-Tomato^{pos} CD45^{pos} cells and CD4^{pos} Foxp3^{pos} (Tregs) among Itgβ68-td-Tomato^{pos} T cells. D-F) Histograms represent the average percentage of T cells among the Itgβ68-tdTomato^{pos} CD45^{pos} cells and the percentage Tregs among the Itgβ8-tdTomato^{pos} T cells in 5 tumors of each. G Transcriptomic analysis from single cell RNAseq data obtained T cell infiltrating human non-small cell lung cancer and human hepatocellular carcinoma. DimPlots and pie-charts show the proportion of Itgβ8-expressing cells among Foxp3^{neg} negative and Foxp3^{pos} T cells for each of the datasets of the different tumors.

FIG. 2: Anti-Itgβ8 antibody specificity.

The specificity of binding of the antibodies was tested by flow cytometry. Mice with deficiency in Itgb8 were immunized Tregs expressing Itgb8. Clones were isolated and their supernatant tested by ELISA. Mouse lymph nodes (LN) cells were incubated with primary isolated clone supernatants positive in ELISA. Conjugated secondary antibody against IgM was used to reveal the clone binding. Histogram illustrated the ability of the antibodies to bind Itgβ8^{pos} versus Itgβ8^{neg} Tregs. Control corresponds to the secondary antibody alone.

FIG. 3: Anti-itgβ8 antibody functionality.

Reporter cells of TGF-β active TLMC were incubated with active TGF-β1 (TGFβ1*), supernatant of Tregs culture (+) for 24 h with inactive TGF-β1 or not and in the presence of supernatant of clones (9B2, 1G10G4, 1G10H5) or control medium (control). Culture condition without Tregs are annotated (−). Graph illustrates the levels of bioactive TGF-β1 using TLMC reporter system of TGF-β activation.

### EXAMPLE

### Material & Methods

### Antibody Generation

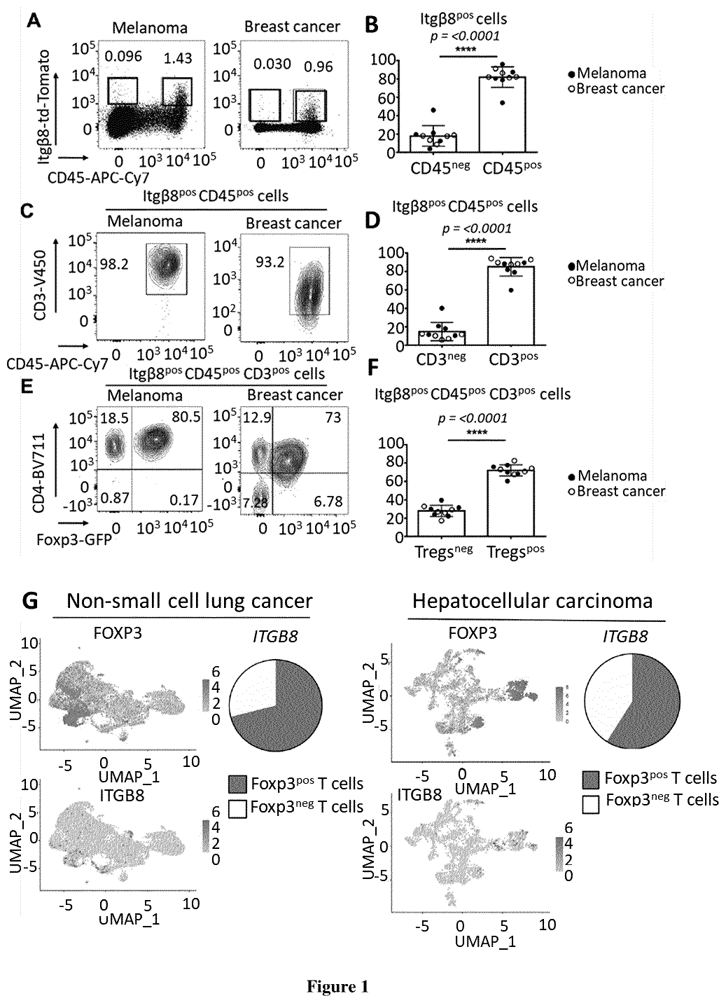

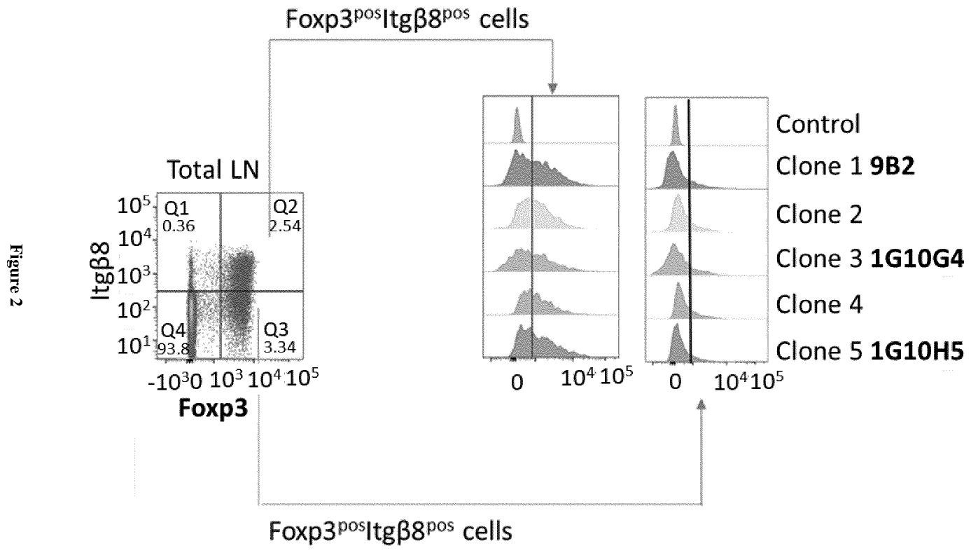

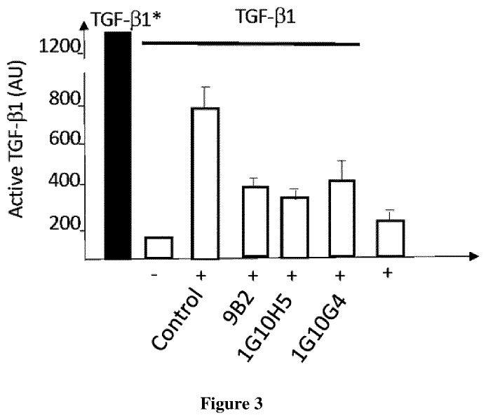

Mice deficient for Itgβ8 were immunized with Tregs, which express Itgβ8. Inventors previously demonstrated that Itgβ8 is largely expressed on Tregs (FIG. 1). 2 million cells were subcutaneously injected in completed Freund Adjuvant. 45 days later mice were rechallenged in the same way. 3 months after the first injection, spleen was harvested and B cell hybridoma established and cloned. Supernatant of hybridoma clones were tested by ELISA using recombinant mouse Itgβ8. 5 clones produced antibodies which recognized Itgβ8.

### Specificity of the Antibodies

In order to address specificity of the 5 clones to Itgβ8 expressed by Tregs, each supernatant was incubated with cells from lymph nodes of Itgβ8 Dt-Tomato reporter, Foxp3^{GFP} mice (depicted in FIG. 1). Cells were analyzed by flow cytometry. The binding was revealed with anti-IgM mouse antibodies. Clearly the antibodies from clone 1, 3, and 5 showed a specific binding to the Tregs cells (Foxp3GFP positive) which expressed Itgβ8 (DtTomato positive).

### Epitope Mapping

Antibodies CDR3 and framework were sequenced. Based on antibody sequences, epitope mapping was established using AlphaFold v2.1.0. Binding sequences were confirmed to be in the extra cellular domain of the Itgβ8 protein. Epitope binding was confirmed to be 100% homologous between mouse and human Itgβ8.

### Functionality

The ability of the antibodies to block the activation of TGF-β1 by Tregs was confirmed by a functional in vitro test. Purified Tregs from draining lymph nodes were incubated with completed RMPI medium in the presence of not 10 ng/mL of inactive TGF-β1 (R&D system) for 24 hours. The supernatants of the Tregs cultures were then applied onto reporter cell line of active TGF-β (TLMC which contain a PAI1 promoter followed by luciferase as disclosed in Lainé et al 2021) for 24h. Luciferase activity was measured and illustrated in arbitrary unit. Activated TGF-β1 (R&D system) was used as positive control.

### Results:

We generated monoclonal antibodies against Itgβ8 which selectively neutralize the ability of Tregs to activate the TGF-β in the TME. Antibodies were generated by immunization of mice deficient in Itgb8 with Tregs expressing Itgb8. Among the 5 clones generated, clone 1, 3 and 5 showed a specific binding to the Tregs cells (Foxp3GFP positive) which expressed Itgβ8 (FIG. 2). These clones were selected and named respectively 9B2, 1G10G4 and 1G10G5.

The ability to these antibodies to block the activation of TGF-β1 by Tregs was then analysed and confirmed (FIG. 3).

Our first analysis on one fresh human colonic tumor revealed a specific binding of the clones on Treg cells (CD3pos CD4pos CD25pos Foxp3pos CD127neg) compared to the other cells in the tumor microenvironment (data not shown). Of note no binding on human blood Treg cells was observed suggesting the specificity of the binding on Treg cells of the tumor microenvironment (data not shown). This observation is in line with previous works revealing a weak expression of the Itgb8 chain on human Treg cells in the blood compared to the tumor. (Plitas G, et al. 2016)

### Conclusion:

In sum, the generated antibodies selectively bind to Itgβ8 of Tregs. Their binding sites are conserved between mice and humans and are localized in the extracellular domain of the protein. As expected the sequences recognized by the antibodies are largely conformational and localized in same conformational region of the protein. The antibodies are endowed with the ability to block the ability of Itgβ8 of Tregs to activate TGF-β1 and thus should prevent from tumor growth by activating the CD8 T cell anti-tumor immune response. Indeed, it has been previously demonstrated that β8 chain of αvβ8 integrin (Itgβ8) expressed on Tregs specifically promotes TGF-β activation and impairs the anti-CD8 T cell response within the TME of different cancers in both mice and humans (Lainé et al, Nature Com, 2017).

This unique specificity to ITGβ8 of Tregs cells makes the antibodies developed herein potent tools to cure cancers and any pathologies associated with Tregs ability to activate TGF-β.

### REFERENCES

Throughout this application, various references describe the state of the art to which this invention pertains. The disclosures of these references are hereby incorporated by reference into the present disclosure.
Batlle E, Massagué J. Transforming Growth Factor-β Signaling in Immunity and Cancer. Immunity. 2019 Apr. 16; 50(4):924-940.
Gao B, Sun W, Wang X, Jia X, Ma B, Chang Y, Zhang W, Xue D. Whole genome expression profiling and screening for differentially expressed cytokine genes in human bone marrow endothelial cells treated with humoral inhibitors in liver cirrhosis. Int J Mol Med. 2013 November; 32(5):1204-14.
Shull M M, Ormsby I, Kier A B, Pawlowski S, Diebold R J, Yin M, Allen R, Sidman C, Proetzel G, Calvin D, et al. Targeted disruption of the mouse transforming growth factor-beta 1 gene results in multifocal inflammatory disease. Nature. 1992 Oct. 22; 359(6397):693-9.
Marie J C, Liggitt D, Rudensky A Y. Cellular mechanisms of fatal early-onset autoimmunity in mice with the T cell-specific targeting of transforming growth factor-beta receptor. Immunity. 2006 September; 25(3):441-54.
Gorelik L, Flavell R A. Immune-mediated eradication of tumors through the blockade of transforming growth factor-beta signaling in T cells. Nat Med. 2001 October; 7(10):1118-22.
Dahmani A, Delisle J S. TGF-β in T Cell Biology: Implications for Cancer Immunotherapy. Cancers (Basel). 2018 Jun. 11; 10(6):194.
Huynh L K, Hipolito C J, Ten Dijke P. A Perspective on the Development of TGF-β Inhibitors for Cancer Treatment. Biomolecules. 2019 Nov. 17; 9(11):743.
Travis M A, Sheppard D. TGF-β activation and function in immunity. Annu Rev Immunol. 2014; 32:51-82.
Courau T, Nehar-Belaid D, Florez L, Levacher B, Vazquez T, Brimaud F, Bellier B, Klatzmann D. TGF-β and VEGF cooperatively control the immunotolerant tumor environment and the efficacy of cancer immunotherapies. JCI Insight. 2016 Jun. 16; 1(9):e85974.
Donkor M K, Sarkar A, Savage P A, Franklin R A, Johnson L K, Jungbluth A A, Allison J P, Li M O. T cell surveillance of oncogene-induced prostate cancer is impeded by T cell-derived TGF-β1 cytokine. Immunity. 2011 Jul. 22; 35(1):123-34.
Lainé, A., Labiad, O., Hemandez-Vargas, H. et al. Regulatory T cells promote cancer immune-escape through integrin αvβ8-mediated TGF-β activation. Nat Commun 12, 6228 (2021).
Lienart S, Merceron R, Vanderaa C, Lambert F, Colau D, Stockis J, van der Woning B, De Haard H, Saunders M, Coulie P G, Savvides S N, Lucas S. Structural basis of latent TGF-β1 presentation and activation by GARP on human regulatory T cells. Science. 2018 Nov. 23; 362(6417):952-956.
Aluwihare, P., Mu, Z., Zhao, Z., Yu, D., Weinreb, P. H., Horan, G. S., Violette, S. M., and Munger, J. S. (2009). Mice that lack activity of alphavbeta6- and alphavbeta8-integrins reproduce the abnormalities of Tgfb1- and Tgfb3-null mice. J Cell Sci 122, 227-232.
Worthington, J. J., Kelly, A., Smedley, C., Bauche, D., Campbell, S., Marie, J. C., and Travis, M. A. (2015). Integrin alphavbeta8-Mediated TGF-beta Activation by Effector Regulatory T Cells Is Essential for Suppression of T-Cell-Mediated Inflammation. Immunity 42, 903-915.
Plitas G, Konopacki C, Wu K, Bos P D, Morrow M, Putintseva E V, Chudakov D M, Rudensky A Y. Regulatory T Cells Exhibit Distinct Features in Human Breast Cancer. Immunity. 2016 Nov. 15; 45(5):1122-1134.

1. An isolated anti-β8 chain of αvβ8 integrin (Itgβ8) neutralizing antibody, wherein said antibody specifically binds to β8 chain of αvβ8 integrin expressed on T regulatory cells.

2. The isolated anti-Itgβ8 neutralizing antibody of claim 1, wherein said antibody comprises
(a) a heavy chain wherein the variable domain comprises:
a H-CDR1 having the following sequence: G-Y-T-F-T-X₆-Y-X₈ (SEQ ID NO:14) wherein X₆ is S or R, and X₈ is T or W;
a H-CDR2 having the following sequence: I-N-P-S-S-G-Y-T (SEQ ID NO:2);
a H-CDR3 having the following sequence: A-R-X₃-E-X₅-X₆-X₇-X₈-X₉-X₁₀-X₁₁-Y-X₁₃-X₁₄-X₁₅-X₁₆-X₁₇ (SEQ ID NO:15) wherein X₃ is A or none, X₅ is G or V, X₆ is L or Y, X₇ is R or Y, X₈ is A or Y, X₉ is W or G, X₁₀ is F or S, X₁₁ is A or S, X₁₃ is G or none, X₁₄ is D or none, X₁₅ is F or none, X₁₆ is D or none and X₁₇ is Y or none; and
(b) a light chain wherein the variable domain comprises:
a L-CDR1 having the following sequence: X₁-X₂-V-X₄-X₅-X₆, wherein X₁ is Q or S, X₂ is N or S, X₄ is G or S, X₅ is T or Y, and X₆ is N or none,
a L-CDR2 having the following sequence: X₁-X₂-S, wherein X₁ is S or R, and X₂ is A or T,
a L-CDR3 having the following sequence: Q-Q-Y-X₄-S-Y-P-X₈-T (SEQ ID NO:16) wherein X₄ is N or H, and X₈ is Y or L.

3. The isolated anti-Itgβ8 neutralizing antibody of claim 1, wherein the antibody comprises (a) a heavy chain wherein the variable domain comprises: a H-CDR1 having a sequence set forth as SEQ ID NO:1 or SEQ ID NO:8; a H-CDR2 having a sequence set forth as SEQ ID NO:2; and a H-CDR3 having a sequence set forth as SEQ ID NO:3 or SEQ ID NO:9; and (b) a light chain wherein the variable domain comprises: a L-CDR1 having a sequence set forth as SEQ ID NO:4 or SEQ ID NO:10; a L-CDRaving a sequence set forth as SAS or RTS; and a L-CDR3 having a sequence set forth as SEQ ID NO:5 or SEQ ID NO:11.

4. The isolated anti-Itgβ8 neutralizing antibody of claim 3, wherein the antibody comprises (a) a heavy chain wherein the variable domain comprises: a H-CDR1 having a sequence set forth as SEQ ID NO:1; a H-CDR2 having a sequence set forth as SEQ ID NO:2; and a H-CDR3 having a sequence set forth as SEQ ID NO:3; and (b) a light chain wherein the variable domain comprises: a L-CDR1 having a sequence set forth as SEQ ID NO:4; a L-CDR2 having a sequence set forth as SAS; and a L-CDR3 having a sequence set forth as SEQ ID NO:5.

5. The isolated anti-Itgβ8 neutralizing antibody of claim 1, wherein the antibody comprises: (a) a heavy chain wherein the variable domain having at least 70% of identity with a sequence set forth as SEQ ID NO:6, and (b) a light chain wherein the variable domain having at least 70% of identity with a sequence set forth as SEQ ID NO:7.

6. The isolated anti-Itgβ8 neutralizing antibody of claim 3, wherein the antibody comprises (a) a heavy chain wherein the variable domain comprises: a H-CDR1 having a sequence set forth as SEQ ID NO:8; a H-CDR2 having a sequence set forth as SEQ ID NO:2; and a H-CDR3 having a sequence set forth as SEQ ID NO:9; and (b) a light chain wherein the variable domain comprises: a L-CDR1 having a sequence set forth as SEQ ID NO:10; a L-CDR2 having a sequence set forth as RTS; and a L-CDR3 having a sequence set forth as SEQ ID NO:11.

7. The isolated anti-Itgβ8 neutralizing antibody of claim 1, wherein the antibody comprises: (a) a heavy chain wherein the variable domain having at least 70% of identity with a sequence set forth as SEQ ID NO:12, and (b) a light chain wherein the variable domain having at least 70% of identity with a sequence set forth as SEQ ID NO:13.

8. The isolated anti-Itgβ8 neutralizing antibody according to claim 1, wherein the antibody is a humanized antibody.

9. A nucleic acid molecule encoding the antibody according to claim 1.

10. A vector that comprises the nucleic acid of claim 9.

11. A host cell which has been transfected, infected or transformed by the nucleic acid of claim 9.

12. A cross-competing antibody which cross-competes for binding β8 chain of αvβ8 integrin expressed on T regulatory cells with the antibody according to claim 1.

13. A pharmaceutical composition comprising i) the anti-Itgβ8 neutralizing antibody according to claim 1 or a fragment thereof and ii) a pharmaceutically acceptable carrier.

14. A method of using the anti-Itgβ8 neutralizing antibody according to claim 1 or a fragment thereof comprising activating the anti-tumoral CD8 T cell response of a subject affected with a cancer with the anti-Itgβ8 neutralizing antibody.

15. A method for treating cancer in a subject in need thereof, comprising administering comprising administering to said subject an effective amount of the anti-Itgβ8 neutralizing antibody according to claim 1 or a fragment thereof.

16. The method according to claim 15, wherein the cancer is selected from the group consisting of glioma, thyroid cancer, colorectal cancer, head and neck cancer, stomach cancer, liver cancer, pancreatic cancer, renal cancer, ovarian cancer, laryngeal squamous cell carcinoma, lung cancer, prostate cancer, cervical cancer, testis cancer, endometrial cancer, breast cancer and melanoma cancer.


SEQUENCE LISTING (20 sequences)

SEQ ID NO: 1 - protein, 8 aa, synthetic construct
GYTFTSYT

SEQ ID NO: 2 - protein, 8 aa, synthetic construct
INPSSGYT

SEQ ID NO: 3 - protein, 12 aa, synthetic construct
ARAEGLRAWFAY

SEQ ID NO: 4 - protein, 6 aa, synthetic construct
QNVGTN

SEQ ID NO: 5 - protein, 9 aa, synthetic construct
QQYNSYPYT

SEQ ID NO: 6 - protein, 119 aa, synthetic construct
QVQLQQSGAELARPGASVKMSCKASGYTFTSYTMHWVKQRPGQGLEWIGYINPSSGYTKYNQKFKDKATLTADKSSSTAYMQLSSLTSEDSAVYYCARAEGLRAWFAYWGQGTLVTVSA

SEQ ID NO: 7 - protein, 107 aa, synthetic construct
DIVMTQSQKFMSTSVGDRVSVTCKASQNVGTNVAWYQQKPGQSPKALIYSASYRYSGVPDHFTGSGSGTDFTLTIINVQSEDLAEYFCQQYNSYPYTFGGGTKLEIK

SEQ ID NO: 8 - protein, 8 aa, synthetic construct
GYTFTRYW

SEQ ID NO: 9 - protein, 16 aa, synthetic construct
AREVYYYGSSYGDFDY

SEQ ID NO: 10 - protein, 5 aa, synthetic construct
SSVSY

SEQ ID NO: 11 - protein, 9 aa, synthetic construct
QQYHSYPLT

SEQ ID NO: 12 - protein, 123 aa, synthetic construct
QVQLQQSGAELAKPGASVKLSCKASGYTFTRYWMHWVKQRPGQGLEWIGHINPSSGYTKYNQKFKDKATLTADKSSSTAYMQLSSLTYEDSAVYYCAREVYYYGSSYGDFDYWGQGTTLTVSS

SEQ ID NO: 13 - protein, 106 aa, synthetic construct
QIVLTQSPAIMSASPGEKVTISCSASSSVSYMYWYQQKPGSSPKPWIYRTSNLASGVPARFSGSGSGTSYSLTISSMEAEDAATYYCQQYHSYPLTFGAGTKLELK

SEQ ID NO: 14 - protein, 8 aa, synthetic construct
  VARIANT 6: note = X is S or R
  VARIANT 8: note = X is T or W
GYTFTXYX

SEQ ID NO: 15 - protein, 17 aa, synthetic construct
  VARIANT 5: note = X is G or V
  VARIANT 6: note = X is L or Y
  VARIANT 7: note = X is R or Y
  VARIANT 8: note = X is A or Y
  VARIANT 9: note = X is W or G
  VARIANT 10: note = X is F or S
  VARIANT 11: note = X is A or S
  VARIANT 13: note = X is G or no amino acid
  VARIANT 14: note = X is D or no amino acid
  VARIANT 15: note = X is F or no amino acid
  VARIANT 16: note = X is D or no amino acid
  VARIANT 17: note = X is Y or no amino acid
  VARIANT 3: note = X is A or no amino acid
ARXEXXXXXXXYXXXXX

SEQ ID NO: 16 - protein, 9 aa, synthetic construct
  VARIANT 4: note = X is N or H
  VARIANT 8: note = X is Y or L
QQYXSYPXT

SEQ ID NO: 17 - DNA, 357 bp, synthetic construct
  source 1..357: mol_type = other DNA
caggtccagctgcagcagtctggggctgaactggcaagacctggtgcctcagtgaagatgtcctgcaaggcttctggctacacctttactagctacacgatgcactgggtaaaacagaggcctggacagggtctggaatggattggatacattaatcctagcagtggttatactaagtacaatcagaagttcaaggacaaggccacattgactgcagacaaatcctccagcacagcctacatgcaactgagcagcctgacatctgaggactctgcagtctattactgtgcaagagctgagggattaagggcctggtttgcttactggggccaagggactctggtcactgtctctgca

SEQ ID NO: 18 - DNA, 321 bp, synthetic construct
  source 1..321: mol_type = other DNA
gacattgtgatgacccagtctcaaaaattcatgtccacatcagtaggagacagggtcagcgtcacctgcaaggccagtcagaatgtgggtactaatgtagcctggtatcagcagaaaccagggcaatctcctaaagcactgatttactcggcatcctaccggtacagtggagtccctgatcacttcacaggcagtggatctgggacagatttcactctcaccatcatcaatgtgcagtctgaagacttggcagagtatttctgtcagcaatataacagctatccgtacacgttcggaggggggaccaagctggaaataaaa

SEQ ID NO: 19 - DNA, 369 bp, synthetic construct
  source 1..369: mol_type = other DNA
caggtccagctgcagcagtctggggctgaactggcaaaacctggggcctcagtgaagctgtcctgcaaggcttctggctacacctttactaggtactggatgcactgggtaaaacagaggcctggacagggtctggaatggattggacacattaatcctagcagtggttatactaagtacaatcagaagttcaaggacaaggccacattgactgcagacaaatcctccagcacagcctacatgcagctgagcagcctgacatatgaggactctgcagtctattactgtgcaagagaggtttattactacggtagtagctacggagactttgactactggggccaaggcaccactctcacagtctcctca

SEQ ID NO: 20 - DNA, 318 bp, synthetic construct
  source 1..318: mol_type = other DNA
caaattgttctcacccagtctccagcaatcatgtctgcatctccaggggagaaggtcaccatatcctgcagtgccagctcaagtgtaagttacatgtactggtaccagcagaagccaggatcctcccccaaaccctggatttatcgcacatccaacctggcttctggagtccctgctcgcttcagtggcagtgggtctgggacctcttactctctcacaatcagcagcatggaggctgaagatgctgccacttattactgccagcagtatcatagttacccactcacgttcggtgctgggaccaagctggagctgaaa